(19) United States
(12) Patent Application Publication
(10) Pub. No.: US 2011/0130302 A1
(43) Pub. Date: Jun. 2, 2011

(54) BIOLOGICAL PATHWAYS ASSOCIATED WITH CHEMOTHERAPY OUTCOME FOR BREAST CANCER

(75) Inventors: Kui SHEN, Pittsburgh, PA (US); Nan Song, Pittsburgh, PA (US); Shara D. Rice, Pittsburgh, PA (US); Dakun Wang, Pittsburgh, PA (US); David A. Gingrich, Pittsburgh, PA (US); Zhenyu Ding, Pittsburgh, PA (US); Chunqiao Tian, Pittsburgh, PA (US); Stacey L. Brower, Pittsburgh, PA (US); Paul R. Ervin, Pittsburgh, PA (US); Michael Gabrin, Pittsburgh, PA (US)

(73) Assignee: Precision Therapeutics, Inc., Pittsburgh, PA (US)

(21) Appl. No.: 12/957,608

(22) Filed: Dec. 1, 2010

Related U.S. Application Data
(60) Provisional application No. 61/265,589, filed on Dec. 1, 2009.

(51) Int. Cl. C40B 30/04; C12Q 1/68; C40B 40/06

(52) U.S. Cl. 506/9; 435/6.11; 506/16

(57) ABSTRACT

The present invention provides methods for preparing drug response and/or resistance profiles for breast tumor specimens, or cells derived therefrom. The drug response and/or resistance profiles are useful for determining effective chemotherapeutic agents for treatment of the tumor or cell to thereby individualize patient therapy. In other aspects, the invention provides a method for identifying a pathway or gene expression signature indicative of a breast cancer cell's sensitivity to a chemotherapeutic agent, which is useful for identifying a population response rate, or patient sub-population likely to respond to the drug candidate.

### PRIORITY

This application claims priority to U.S. Provisional Application No. 61/265,589, filed Dec. 1, 2009, which is hereby incorporated by reference.

### BACKGROUND

There are several approaches to improving cancer chemotherapeutic treatment. One approach seeks to understand the biochemical pathways and coding genes involved in cancer causation to improve drug candidates, while another seeks to understand the biochemical pathways involved in drug response to determine who will respond to a given drug.

Chemotherapy response pathways can provide important information for studying drug resistance mechanisms, and have diagnostic and/or prognostic utility for individualizing patient therapy. Riedel et al., A genomic approach to identify molecular pathways associated with chemotherapy resistance, Mol. Cancer Ther. 7(10):3141-3149 2008; Adewale A J et al., Pathway analysis of microarray data via regression, J. of Computational Biology 15(3):269-277 (2008); Bild et al., Linking oncogenic pathways with therapeutic opportunity, Nature Reviews Vol. 6 (September 2006). Identification of pathway and gene expression signatures indicative of response and/or resistance to chemotherapeutic agents provides the ability to improve therapeutic efficacy by gene-expression analysis of patient tumors and/or malignant cells.

### SUMMARY OF THE INVENTION

The present invention provides methods for preparing drug response and/or resistance profiles for breast tumor specimens, or cells derived therefrom. The drug response and/or resistance profiles are useful for determining effective chemotherapeutic agents or combinations thereof for treatment of the tumor or cell. The drug response and/or resistance profile comprises the expression levels for genes associated with biochemical pathways that are enriched in drug sensitive or drug resistant cells.

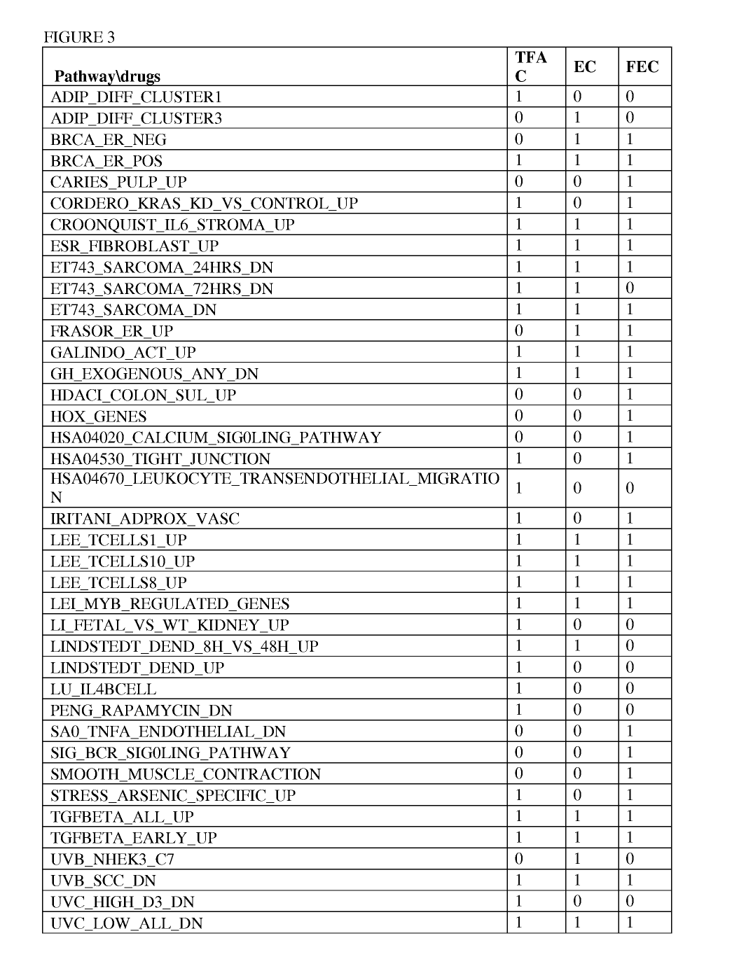

In certain aspects, the invention provides a method for determining a drug response profile for a breast tumor specimen or a cell culture derived therefrom. The method comprises isolating RNA from the tumor specimen or cell culture derived therefrom, and preparing a gene expression profile suitable for pathway analysis as described herein, or for determining the presence of a gene expression signature described herein. The gene expression profile is then evaluated for the presence of one or more pathway signatures or gene expression signatures indicative of response to one or more chemotherapeutic agents or combinations. In certain embodiments, the RNA is isolated from a monolayer culture derived from explants of the breast tumor specimen, or alternatively, is isolated from the specimen itself. Pathway signatures indicative of senstivity or resistance to TFAC, EC, and FEC are disclosed in FIG. 3. Gene expression signatures, which are derived from the pathway analysis, and which are indicative of response to TFAC, EC, and FEC, are provided in Tables 1-3, respectively. The method in certain embodiments is useful for individualizing chemotherapy for a patient, by determining a tumors likely response to a plurality of chemotherapeutic agents or combinations of agents prior to treatment.

In other aspects, the invention provides a method for determining a pathway signature or gene expression signature for a chemotherapeutic agent for breast cancer. The method comprises determining the level of sensitivity of a panel of breast cancer cell lines for the chemotherapeutic agent in vitro, and evaluating the gene expression levels of the breast cancer cell lines to identify biochemical pathways associated with the drug-sensitive and/or drug-resistant cell lines (“enriched pathways”). Generally, the enriched pathways are those having a significant number of associated genes being differentially expressed in the drug sensitive or drug resistant populations. In certain embodiments, the panel of breast cancer cell lines are immortalized cell lines. In other embodiments, the panel of breast cancer cell lines are derived from explants of patient tumor specimens, and are further useful for identifying a population response rate, or patient sub-population likely to respond to the drug candidate. In still other embodiments, the enriched pathways are evaluated to identify and/or select genes that are differentially expressed in drug sensitive versus drug resistant cells, to thereby identify discrete gene expression signatures associated with drug response.

In still other aspects, the invention provides kits and probe sets useful for determining patient profiles that are indicative of a tumor's sensitivity or resistance to certain chemotherapeutic agents or combinations.

The present application discloses biological pathways associated with resistance to three drug combinations in breast cancer cell lines. Specifically, 43 immortalized breast cancer cell lines were exposed to three drug combinations separately using the CHEMOFX protocol. Area under the dose-response curve (AUC) was calculated to measure the chemosensitivity. By comparing public breast cancer cell line microarray data and biological pathway databases (Biocarta Pathway Collections, biocarta.com/genes/allPathwaysasp; Kanehisa M,Goto S, KEGG: Kyoto Encyclopedia of Genes and Genomes Nucl. Acids Res. 28(1):27-30 2000) with the measured chemotherapeutic response, specific biochemical pathways associated with response to each of the 3 drug combinations were identified. These drug response-associated pathways are valuable for new biomarker identification and discovering new drug targets, as well as individualizing patient therapy.

### DETAILED DESCRIPTION OF THE FIGURES

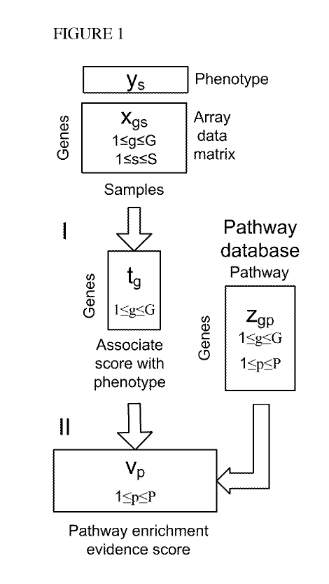

FIG. 1 illustrates a pathway enrichment algorithm.

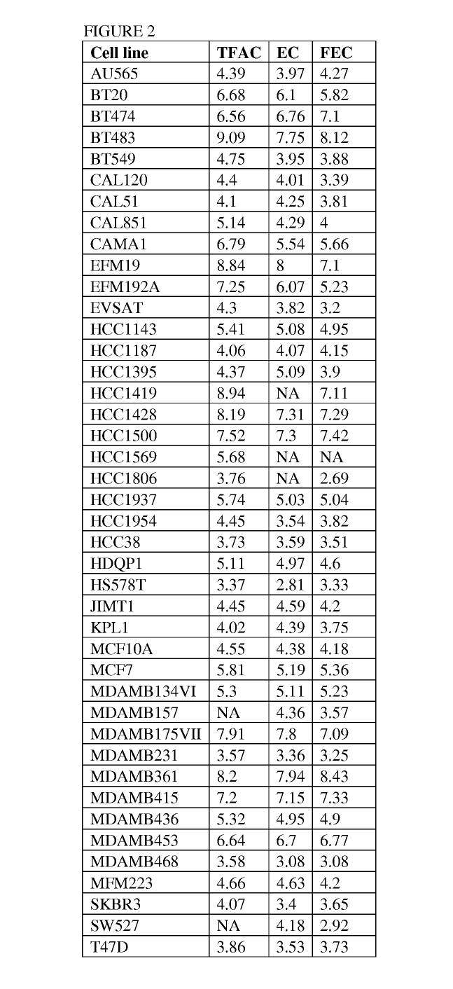

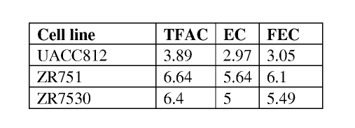

FIG. 2 lists the AUC values for breast cancer cell lines tested using the CHEMOFX assay. The heading row lists the drug or combination tested.

FIG. 3 shows the enriched pathways for the 3 drug combinations tested.

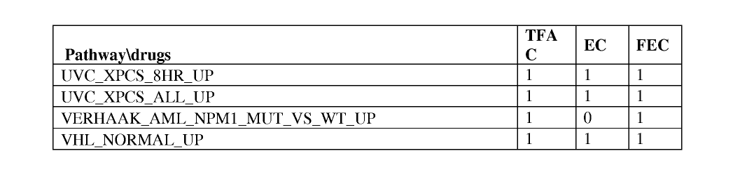

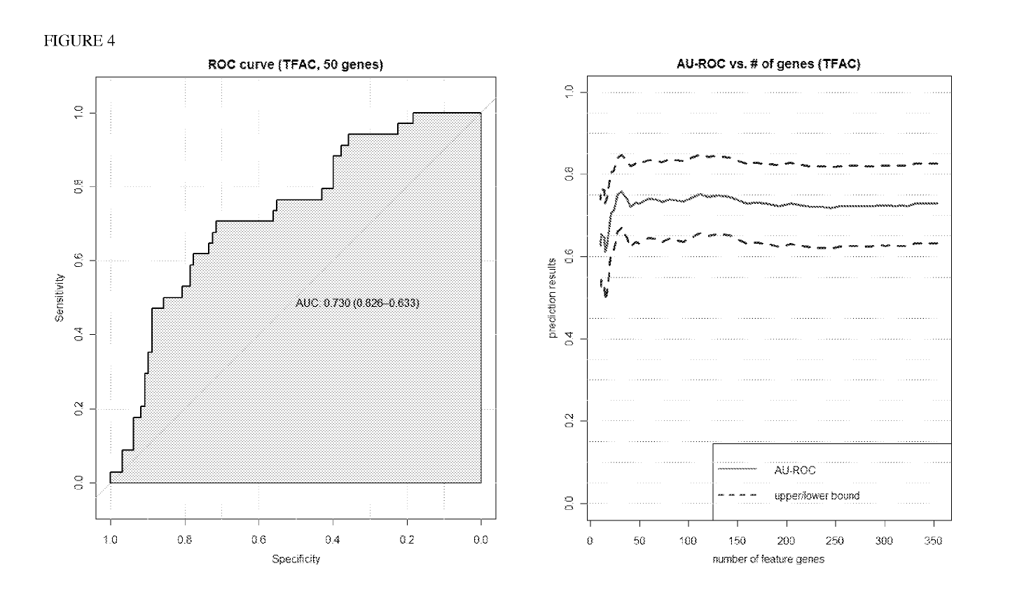

FIG. 4 illustrates the accuracy of a 50-gene signature from Table 1 for predicting pCR in an independent patient population (133 neoadjuvant breast cancer patients treated with TFAC). Outcome is pathological complete response (pCR). The results are shown as a receiver operator curve (ROC). When using one third of the prediction scores as cutoff, the accuracy is 0.73, sensitivity is 0.62 and specificity is 0.78. The right panel shows that the gene expression signature of Table 1 is stable over a large range of increasing gene number, from less than about 10 to over 350 genes (Table 1 lists the top 50 genes).

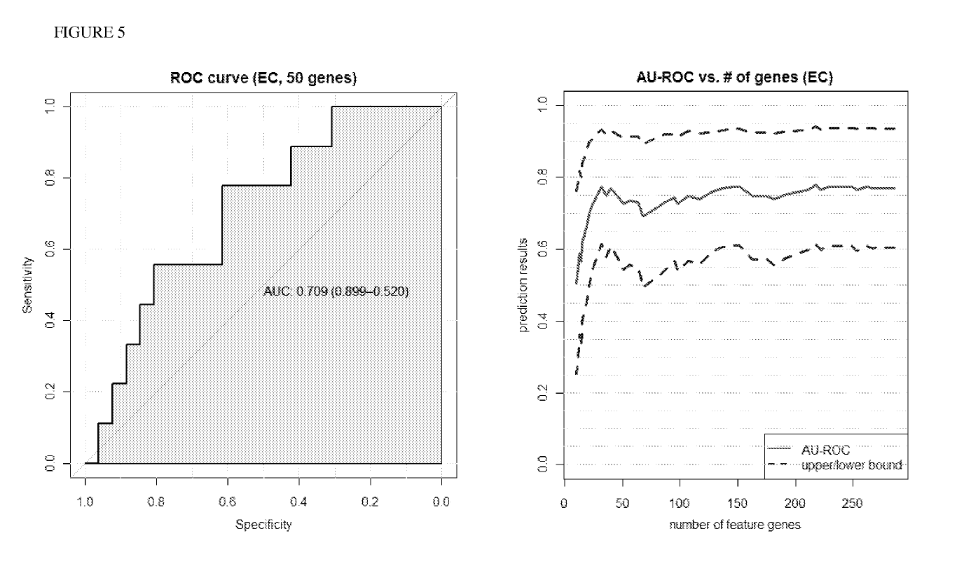

FIG. 5 illustrates the accuracy of a 50-gene signature from Table 2 for predicting pCR in an independent patient population (37 neoadjuvant breast cancer patients treated with EC). Outcome is pathological complete response (pCR). The results are shown as a receiver operator curve (ROC). When using one third of the prediction scores as cutoff, the accuracy is 0.71, sensitivity is 0.56 and specificity is 0.77.The right panel shows that the gene expression signature of Table 3 is stable over a large range of increasing gene number, from less than about 10 to over 250 genes (Table 2 lists the top 50 genes).

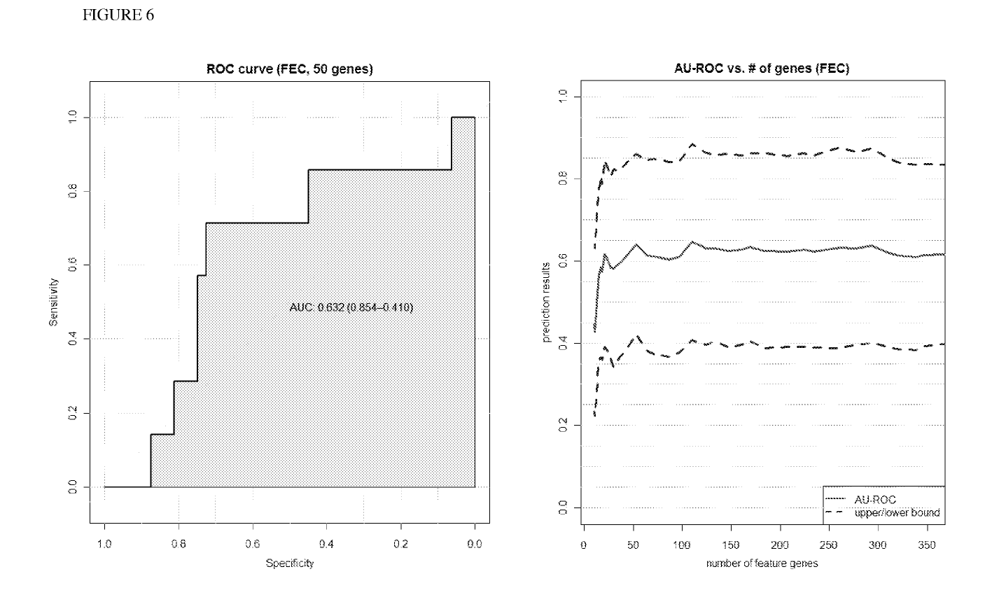

FIG. 6 illustrates the accuracy of a 50-gene signature from Table 3 for predicting pCR in an independent patient population (87 neoadjuvant breast cancer patients treated with FAC). Outcome is pathological complete response (pCR). The results are shown as a receiver operator curve (ROC). When using one third of the prediction scores as cutoff, the accuracy is 0.71, sensitivity is 0.71 and specificity is 0.71. The right panel shows that the gene expression signature of Table 3 is stable over a large range of increasing gene number, from less than about 10 to over 350 genes (Table 3 lists the top 50 genes).

### DETAILED DESCRIPTION OF THE INVENTION

The invention provides a method for determining a drug response profile for a breast tumor specimen or a cell culture derived therefrom, where the drug response profile may be evaluated for the presence of one or more pathway or gene expression signatures indicative of sensitivity and/or resistance to one or more chemotherapeutic agents or combinations. In other aspects, the invention provides methods for identifying such pathway and gene expression signatures.

### Drug Response Profiles and Signatures

The patient generally is a breast cancer patient, and the tumor is generally a solid tumor of epithelial origin. The tumor specimen may be obtained from the patient by surgery, or may be obtained by biopsy, such as a fine needle biopsy or other procedure prior to the selection/initiation of therapy. In certain embodiments, the breast cancer is preoperative or post-operative breast cancer. In certain embodiments, the patient has not undergone treatment to remove the breast tumor, and therefore is a candidate for neoadjuvant therapy.

The cancer may be primary or recurrent, and may be of any type (as described above), stage (e.g., Stage I, II, III, or IV or an equivalent of other staging system), and/or histology. The patient may be of any age, sex, performance status, and/or extent and duration of remission.

In certain embodiments, the patient is a candidate for treatment with one or more of cyclophosphamide, docetaxel, doxorubicin, fluorouracil, epirubicin, or paclitaxel, or a combination thereof. For example, the patient may be a candidate for treatment with TFAC (paclitaxel, 5-fluorouracil, doxorubicin, and cyclophosphamide), EC (epirubicin and cyclphosphamide), or FEC (5-fluorouracil, epirubicin, and cyclophosphamide).

A gene expression profile is determined for the tumor tissue or cell sample, such as a tumor sample removed from the patient by surgery or biopsy. The tumor sample may be “fresh,” in that it was removed from the patent within about five days of processing, and remains suitable or amenable to culture. In some embodiments, the tumor sample is not “fresh,” in that the sample is not suitable or amenable to culture. Tumor samples are generally not fresh after from 3 to 7 days (e.g., about five days) of removal from the patient. The sample may be frozen after removal from the patient, and preserved for later RNA isolation. The sample for RNA isolation may be a formalin-fixed paraffin-embedded (FFPE) tissue.

In certain embodiments, the malignant cells are enriched or expanded in culture by forming a monolayer culture from tumor sample explants. For example, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant.

For example, where it is desirable to expand and/or enrich malignant cells in culture relative to non-malignant cells that reside in the tumor, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³. After the tissue sample has been minced, the particles are plated in culture flasks. The number of explants plated per flask may vary, for example, between one and 25, such as from 5 to 20 explants per flask. For example, about 9 explants may be plated per T-25 flask, and 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of days to a few weeks a cell monolayer will form.

Further, it is believed that tumor cells grow out from the multicellular explant prior to stromal cells. Thus, by initially maintaining the tissue cells within the explant and removing the explant at a predetermined time (e.g., at about 10 to about 50 percent confluency, or at about 15 to about 25 percent confluency), growth of the tumor cells (as opposed to stromal cells) into a monolayer is facilitated. In certain embodiments, the tumor explant may be agitated to substantially loosen or release tumor cells from the tumor explant, and the released cells cultured to produce a cell culture monolayer. The use of this procedure to form a cell culture monolayer helps maximize the growth of representative malignant cells from the tissue sample. Monolayer growth rate and/or cellular morphology (e.g., epithelial character) may be monitored using, for example, a phase-contrast inverted microscope. Generally, the cells of the monolayer should be actively growing at the time the cells are suspended for RNA extraction.

The process for enriching or expanding malignant cells in culture is described in U.S. Pat. Nos. 5,728,541, 6,900,027, 6,887,680, 6,933,129, 6,416,967, 7,112,415, 7,314,731, 7,642,048 and 7,501,260 (all of which are hereby incorporated by reference in their entireties). The process may further employ the variations described in US Published Patent Application No. 2007/0059821, which is hereby incorporated by reference its entirety.

In preparing gene expression profiles, RNA is extracted from the tumor tissue or cultured cells by any known method. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, there are various products commercially available for RNA isolation which may be used. Total RNA or polyA+ RNA may be used for preparing gene expression profiles in accordance with the invention.

The gene expression profile is then generated for the samples using any of various techniques known in the art. Such methods generally include, without limitation, hybridization-based assays, such as microarray analysis and similar formats (e.g., Whole Genome DASL™ Assay, Illumina, Inc.), polymerase-based assays, such as RT-PCR (e.g., Taqman™), flap-endonuclease-based assays (e.g., Invader™), as well as direct mRNA capture with branched DNA (QuantiGene™) or Hybrid Capture™ (Digene). The method may or may not employ amplification steps. In certain embodiments, the profile is generated using an HG-U133 chip (e.g., 2.0 array) or comparable microarray.

The gene expression profile contains gene expression levels for a plurality of genes whose expression levels are predictive or indicative of the tumor's resistance to one or a combination of chemotherapeutic agents. Such genes are associated with the enriched pathways disclosed in FIG. 3, and/or are listed in one of Tables 1-3. Genes associated with the enriched pathways are known and publicly available, for example, at Biocarta Pathway Collections, biocarta.com/genes/allPathwaysasp; Kanehisa M,Goto S, KEGG: Kyoto Encyclopedia of Genes and Genomes Nucl. Acids Res. 28(1):27-30 2000).

As used herein, the term “gene,” refers to a DNA sequence expressed in a sample as an RNA transcript, and may be a full-length gene (protein encoding or non-encoding) or an expressed portion thereof such as expressed sequence tag or “EST.” Thus, the genes associated with enriched pathways and/or listed in Tables 1-3 are each independently a full-length gene sequence, whose expression product is present in samples, or is a portion of an expressed sequence detectable in samples, such as an EST sequence. These gene sequences as well as probe IDs for the HG-U133 Plus 2.0 Chip are known, are publicly available, and are hereby incorporated by reference.

The genes associated with the enriched biochemical pathways may be differentially expressed in drug-sensitive samples versus drug-resistant samples. As used herein, “differentially expressed” means that the level or abundance of an RNA transcript (or abundance of an RNA population sharing a common target (or probe-hybridizing) sequence, such as a group of splice variant RNAs) is significantly higher or lower in a drug-sensitive sample as compared to a reference level (e.g., in a non-responsive sample). For example, the level of the RNA or RNA population may be higher or lower than a reference level. The reference level may be the level of the same RNA or RNA population in a control sample or control population (e.g., a Mean or Median level for a non-responsive sample), or may represent a cut-off or threshold level for a sensitive or resistant designation.

The gene expression profile generally contains the expression levels for a sufficient number of genes to perform pathway analysis or evaluate for the presence of a gene expression signature as described herein.

For example, the gene expression profile may contain the expression levels for at least about 10, 25, 50, 100, 500, 1000 genes or more, with these genes being associated with the enriched pathways disclosed herein. The profile may comprise the expression level of at least 10, 20, 30, 40, or 50 genes listed in any one of Tables 1-3. Where a significant number of genes associated with a pathway are differentially expressed, the pathway is deemed an “enriched pathway.” In some embodiments, the profile is prepared with the use of a custom array or bead set (or other gene expression detection format), so as to quantify the level of 500 genes of less, 250 genes or less, 150 genes or less, or 100 genes or less, including 3, 5, 7, 10, 25, or 50 genes listed in one of Tables 1-3. In certain embodiments, the custom array or bead set employs corresponding probes from the HG-U133 array (e.g., plus 2.0 Chip).

The pathway and gene expression signature(s) (e.g., data for pathway analysis) may be in a format consistent with any nucleic acid detection format, such as those described herein, and will generally be comparable to the format used for profiling patient samples. For example, the gene expression signatures and patient profiles may both be prepared by nucleic acid hybridization method, and with the same hybridization platform and controls so as to facilitate comparisons. The gene expression signatures may further embody any number of statistical measures, including Mean or median expression levels and/or cut-off or threshold values.

Once the gene expression profile for patient samples are prepared, the profile is evaluated for the presence of one or more of the pathway or gene expression signatures, by scoring or classifying the patient profile against each pathway or gene expression signature. Exemplary pathway signatures for sensitivity or resistance to TFAC, EC, and FEC are disclosed herein in FIG. 3. Exemplary gene expression signatures, derived from the identified enriched pathways, are disclosed in Tables 1-3.

In certain embodiments, the gene expression profile is evaluated for enrichment of one, two, three, five, ten, twenty or more pathways disclosed in FIG. 3 (“pathway signature”). The set of enriched pathways for each signature may be indicative of response to TFAC as set forth in FIG. 3, or may be indicative of a response to EC as set forth in FIG. 3, or may be indicative of response to FEC as disclosed in FIG. 3. Generally, an enriched pathway is identified as a pathway having a significant number of genes differentially expressed in drug sensitive versus drug resistant cells, and determination of pathway enrichment may be conducted based upon the methods and algorithms disclosed herein.

In other embodiments, the gene expression profile is evaluated for the presence of a gene expression signature disclosed in Tables 1-3. The gene expression signatures of Tables 1-3 were derived from the enriched pathways of FIG. 3. Thus, the signature may involve the mean, median, or other measure of expression for 5, 10, 20, or 50 genes listed in Table 1. Such levels of expression are indicative of senstivity to TFAC. In other embodiments, the signature may involve the mean, median, or other measure of expression for 5, 10, 20, or 50 genes listed in Table 2. Such levels of expression are indicative of senstivity to EC. In still other embodiments, the signature may involve the mean, median, or other measure of expression for 5, 10, 20, or 50 genes listed in Table 3. Such levels of expression are indicative of senstivity to FEC. By evaluating the gene expression profiles for the presence or absence of these gene expression signatures, the profiles may be classified as sensitive or resistant to TFAC, EC, and/or FEC.

Various classification schemes are known for classifying samples between two or more classes or groups, and these include, without limitation: Principal Components Analysis, Naïve Bayes, Support Vector Machines, Nearest Neighbors, Decision Trees, Logistic, Artificial Neural Networks, and Rule-based schemes. In addition, the predictions from multiple models can be combined to generate an overall prediction. For example, a “majority rules” prediction may be generated from the outputs of a Naïve Bayes model, a Support Vector Machine model, and a Nearest Neighbor model.

Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604, which review is hereby incorporated by reference in its entirety.

Generally, the gene expression profiles for patient specimens are scored or classified as drug-sensitive signatures or drug-resistant signatures using the pathway analysis or gene expression signatures, including with stratified or continuous intermediate classifications or scores reflective of drug resistance or sensitivity. As discussed, such signatures may be assembled from gene expression data disclosed herein, or prepared from independent data sets. The signatures may be stored in a database and correlated to patient tumor gene expression profiles in response to user inputs.

After comparing the patient's gene expression profile to the drug-sensitive and/or drug-resistant signature, the sample is classified as, or for example, given a probability of being, a drug-sensitive profile or a drug-resistant profile. The classification may be determined computationally based upon known methods as described above. The result of the computation may be displayed on a computer screen or presented in a tangible form, for example, as a probability (e.g., from 0 to 100%) of the patient responding to a given treatment. The report will aid a physician in selecting a course of treatment for the cancer patient. For example, in certain embodiments of the invention, the patient's gene expression profile will be determined to be a drug-sensitive profile on the basis of a probability, and the patient will be subsequently treated with that drug or combination. In other embodiments, the patient's profile will be determined to be a drug-resistant profile, thereby allowing the physician to exclude one or more candidate treatments for the patient, thereby sparing the patient the unnecessary toxicity.

The method according to this aspect may lend additional or alternative predictive value over standard methods, such as for example, gene expression tests known in the art, or chemoresponse testing.

The methods of the invention aid the prediction of an outcome of treatment. That is, the gene expression signatures are each predictive of an outcome upon treatment with a candidate agent or combination. The outcome may be quantified in a number of ways. For example, the outcome may be an objective response, a clinical response, or a pathological response to a candidate treatment. The outcome may be determined based upon the techniques for evaluating response to treatment of solid tumors as described in Therasse et al., New Guidelines to Evaluate the Response to Treatment in Solid Tumors, J. of the National Cancer Institute 92(3):205-207 (2000), which is hereby incorporated by reference in its entirety. For example, the outcome may be survival (including overall survival or the duration of survival), progression-free interval, or survival after recurrence. The timing or duration of such events may be determined from about the time of diagnosis or from about the time treatment (e.g., chemotherapy) is initiated. Alternatively, the outcome may be based upon a reduction in tumor size, tumor volume, or tumor metabolism, or based upon overall tumor burden, or based upon levels of serum markers especially where elevated in the disease state. The outcome in some embodiments may be characterized as a complete response, a partial response, stable disease, and progressive disease, as these terms are understood in the art.

In certain embodiments, the gene signature is indicative of a pathological complete response upon treatment with a particular candidate agent or combination (as already described). A pathological complete response, e.g., as determined by a pathologist following examination of tissue (e.g., breast or nodes in the case of breast cancer) removed at the time of surgery, generally refers to an absence of histological evidence of invasive tumor cells in the surgical specimen.

The present invention may further comprise conducting chemoresponse testing with a panel of chemotherapeutic agents on cultured cells from a cancer patient, to thereby add additional predictive value. That is, the presence of one or more indicative pathway signatures, and the in vitro chemoresponse results for the tumor specimen, are used to predict an outcome of treatment (e.g., survival, pCR, etc.). For example, where the gene expression profile and chemoresponse test both indicate that a tumor is sensitive or resistant to a particular treatment, the predictive value of the method may be particularly high. Chemoresponse testing may be performed via the CHEMOFX test, as described herein and as known in the art.

In other aspects, the invention provides a method for identifying a pathway signature indicative of a breast cancer cell or cell line's sensitivity or resistance against a chemotherapeutic agent. The method comprises determining the level of sensitivity of a panel of breast cancer cell lines for the chemotherapeutic agent in vitro, and evaluating the gene expression levels of the breast cancer cell lines to identify biochemical pathways associated with the level of sensitivity. In certain embodiments, the panel of breast cancer cell lines are immortalized cell lines, and may comprise the panel described herein or a subset thereof. In other embodiments, the panel of breast cancer cell lines are derived from explants of patient tumor specimens as described herein (e.g., via ChemoFx), and are useful for identifying a population response rate, or patient sub-population likely to respond to the drug candidate.

### Chemoresponse Assay

The present invention may further comprise conducting chemoresponse testing with a panel of chemotherapeutic agents on cultured cells from the cancer patient, to thereby add additional predictive value. That is, the presence of one or more pathway or gene expression signatures in tumor cells, and the in vitro chemoresponse results for the tumor specimen, are used to predict an outcome of treatment (e.g., survival, pCR, etc.). For example, where the gene expression profile and chemoresponse test both indicate that a tumor is sensitive or resistant to a particular treatment, the predictive value of the method may be particularly high.

Several in vitro chemoresponse systems are known and art, and some are reviewed in Fruehauf et al., In vitro assay-assisted treatment selection for women with breast or ovarian cancer, Endocrine-Related Cancer 9: 171-82 (2002). In certain embodiments, the chemoresponse assay is as described in U.S. Pat. Nos. 5,728,541, 6,900,027, 6,887,680, 6,933,129, 6,416,967, 7,112,415, 7,314,731, 7,501,260 (all of which are hereby incorporated by reference in their entireties). The chemoresponse method may further employ the variations described in US Published Patent Application Nos. 2007/0059821 and 2008/0085519, both of which are hereby incorporated by reference in their entireties.

Briefly, in certain embodiments, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant. Some enzymatic digestion may take place in certain embodiments. Generally, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm3, for example, about 1 mm3.

After the tissue sample has been minced, the particles are plated in culture flasks. The number of explants plated per flask may vary, for example, between one and 25, such as from 5 to 20 explants per flask. For example, about 9 explants may be plated per T-25 flask, and 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of days to a few weeks a cell monolayer will form. Further, it is believed (without any intention of being bound by the theory) that tumor cells grow out from the multicellular explant prior to stromal cells. Thus, by initially maintaining the tissue cells within the explant and removing the explant at a predetermined time (e.g., at about 10 to about 50 percent confluency, or at about 15 to about 25 percent confluency), growth of the tumor cells (as opposed to stromal cells) into a monolayer is facilitated. In certain embodiments, the tumor explant may be agitated to substantially release tumor cells from the tumor explant, and the released cells cultured to produce a cell culture monolayer. The use of this procedure to form a cell culture monolayer helps maximize the growth of representative tumor cells from the tissue sample.

Prior to the chemotherapy assay, the growth of the cells may be monitored, and data from periodic counting may be used to determine growth rates which may or may not be considered parallel to growth rates of the same cells in vivo in the patient. If growth rate cycles can be documented, for example, then dosing of certain active agents can be customized for the patient. Monolayer growth rate and/or cellular morphology may be monitored using, for example, a phase-contrast inverted microscope. Generally, the cells of the monolayer should be actively growing at the time the cells are suspended and plated for drug exposure. The epithelial character of the cells may be confirmed by any number of methods. Thus, the monolayers will generally be non-confluent monolayers at the time the cells are suspended for drug exposure.

A panel of active agents may then be screened using the cultured cells. Generally, the agents are tested against the cultured cells using plates such as microtiter plates. For the chemosensitivity assay, a reproducible number of cells is delivered to a plurality of wells on one or more plates, preferably with an even distribution of cells throughout the wells. For example, cell suspensions are generally formed from the monolayer cells before substantial phenotypic drift of the tumor cell population occurs. The cell suspensions may be, without limitation, about 4,000 to 12,000 cells/ml, or may be about 4,000 to 9,000 cells/ml, or about 7,000 to 9,000 cells/ml. The individual wells for chemoresponse testing are inoculated with the cell suspension, with each well or “segregated site” containing about 10² to 10⁴ cells. The cells are generally cultured in the segregated sites for about 4 to about 30 hours prior to contact with an agent.

Each test well is then contacted with at least one pharmaceutical agent, for example, an agent for which a gene expression signature is available. Such agents include the combination of cyclophosphamide, doxorubicin, fluorouracil, and paclitaxel (“TFAC”), the combination of cyclophosphamide and epirubicin (“EC”), or the combination of cyclophosphamide, epirubicin, fluorouracil (“TFEC”).

The efficacy of each agent in the panel is determined against the patient's cultured cells, by determining the viability of the cells (e.g., number of viable cells). For example, at predetermined intervals before, simultaneously with, or beginning immediately after, contact with each agent or combination, an automated cell imaging system may take images of the cells using one or more of visible light, UV light and fluorescent light. Alternatively, the cells may be imaged after about 25 to about 200 hours of contact with each treatment. The cells may be imaged once or multiple times, prior to or during contact with each treatment. Of course, any method for determining the viability of the cells may be used to assess the efficacy of each treatment in vitro.

In this manner the in vitro efficacy grade for each agent in the panel may be determined. While any grading system may be employed (including continuous or stratified), in certain embodiments the grading system is stratified, having from 2 or 3, to 10 response levels, e.g., about 3, 4, or 5 response levels. For example, when using three levels, the three grades may correspond to a responsive grade (e.g., sensitive), an intermediate responsive grade, and a non-responsive grade (e.g., resistant), as discussed more fully herein. In certain embodiments, the patient's cells show a heterogeneous response across the panel of agents, making the selection of an agent particularly crucial for the patient's treatment.

The output of the assay is a series of dose-response curves for tumor cell survivals under the pressure of a single or combination of drugs, with multiple dose settings each (e.g., ten dose settings). To better quantify the assay results, the invention employs in some embodiments a scoring algorithm accommodating a dose-response curve. Specifically, the chemoresponse data are applied to an algorithm to quantify the chemoresponse assay results by determining an adjusted area under curve (aAUC).

However, since a dose-response curve only reflects the cell survival pattern in the presence of a certain tested drug, assays for different drugs and/or different cell types have their own specific cell survival pattern. Thus, dose response curves that share the same aAUC value may represent different drug effects on cell survival. Additional information may therefore be incorporated into the scoring of the assay. In particular, a factor or variable for a particular drug or drug class (such as those drugs and drug classes described) and/or reference scores may be incorporated into the algorithm. For example, in certain embodiments, the invention quantifies and/or compares the in vitro sensitivity/resistance of cells to drugs having varying mechanisms of action, and thus, in some cases, different dose-response curve shapes. In these embodiments, the invention compares the sensitivity of the patient's cultured cells to a plurality of agents that show some effect on the patient's cells in vitro (e.g., all score sensitive to some degree), so that the most effective agent may be selected for therapy. In such embodiments, an aAUC can be calculated to take into account the shape of a dose response curve for any particular drug or drug class. The aAUC takes into account changes in cytotoxicity between dose points along a dose-response curve, and assigns weights relative to the degree of changes in cytotoxicity between dose points. For example, changes in cytotoxicity between dose points along a dose-response curve may be quantified by a local slope, and the local slopes weighted along the dose-response curve to emphasize cytotoxicity.

For example, aAUC may be calculated as follows.

Step 1: Calculate Cytotoxity Index (CI) for each dose, where CI=Mean_{drug}/Mean_{control}.

Step 2: Calculate local slope (S_{d}) at each dose point, for example, as S_{d}=(CI_{d}-CI_{d-1})/Unit of Dose, or S_{d}=(CI_{d-1}−CI_{d})/Unit of Dose.

Step 3: Calculate a slope weight at each dose point, e.g., W_{d}=1−S_{d}.

Step 4: Compute aAUC, where aAUC=Σ W_{d} CI_{d}, and where, d=1, 2, . . . , 10; aAUC˜(0, 10); And at d=1, then CI_{d-1}=1. Equation 4 is the summary metric of a dose response curve and may used for subsequent regression over reference outcomes.

Usually, the dose-response curves vary dramatically around middle doses, not in lower or higher dose ranges. Thus, the algorithm in some embodiments need only determine the aAUC for a middle dose range, such as for example (where from 8 to 12 doses are experimentally determined, e.g., about 10 doses), the middle 4, 5, 6, or 8 doses are used to calculate aAUC. In this manner, a truncated dose-response curve might be more informative in outcome prediction by eliminating background noise.

The numerical aAUC value (e.g., test value) may then be evaluated for its effect on the patient's cells. For example, a plurality of drugs may be tested, and aAUC determined as above for each, to determine whether the patient's cells have a sensitive response, intermediate response, or resistant response to each drug.

In some embodiments, each drug is designated as, for example, sensitive, or resistant, or intermediate, by comparing the aAUC test value to one or more cut-off values for the particular drug (e.g., representing sensitive, resistant, and/or intermediate aAUC scores for that drug). The cut-off values for any particular drug may be set or determined in a variety of ways, for example, by determining the distribution of a clinical outcome within a range of corresponding aAUC reference scores. That is, a number of patient tumor specimens are tested for chemosenstivity/resistance (as described herein) to a particular drug prior to treatment, and aAUC quantified for each specimen. Then after clinical treatment with that drug, aAUC values that correspond to a clinical response (e.g., sensitive) and the absence of significant clinical response (e.g., resistant) are determined. Cut-off values may alternatively be determined from population response rates. For example, where a patient population is known to have a response rate of 30% for the tested drug, the cut-off values may be determined by assigning the top 30% of aAUC scores for that drug as sensitive. Further still, cut-off values may be determined by statistical measures.

In other embodiments, the aAUC scores may be adjusted for drug or drug class. For example, aAUC values for dose response curves may be regressed over a reference scoring algorithm adjusted for test drugs. The reference scoring algorithm may provide a categorical outcome, for example, sensitive (s), intermediate sensitive (i) and resistant (r), as already described. Logistic regression may be used to incorporate the different information, i.e., three outcome categories, into the scoring algorithm. However, regression can be extended to other forms, such as linear or generalized linear regression, depending on reference outcomes. The regression model may be fitted as the following: Logit (Pref)=α+β (aAUC)+γ (drugs), where γ is a covariate vector and the vector can be extended to clinical and genomic features. The score may be calculated as Score=β (aAUC)+γ (drugs). Since the score is a continuous variable, results may be classified into clinically relevant categories, i.e., sensitive (S), intermediate sensitive (I), and resistant (R), based on the distribution of a reference scoring category or maximized sensitivity and specificity relative to the reference.

As stated, the chemoresponse score for cultures derived from patient specimens may provide additional predictive or prognostic value in connection with the gene expression profile analysis.

Alternatively, where applied to immortalized cell line collections or patient-derived cultures, the in vitro chemoresponse assay may be used to supervise or train pathway and gene expression signatures. Once gene expression signatures are identified in cultured cells, e.g., by correlating the level of in vitro chemosensitivity with gene expression levels, the resulting gene expression signatures may be independently validated in patient test populations having available gene expression data and corresponding clinical data, including information regarding the treatment regimen and outcome of treatment. This aspect of the invention reduces the length of time and quantity of patient samples needed for identifying and validating such gene expression signatures.

### Gene Expression Assay Formats

Gene expression profiles, including patient gene expression profiles and the drug-sensitive and drug-resistant signatures as described herein, may be prepared according to any suitable method for measuring gene expression. That is, the profiles may be prepared using any quantitative or semi-quantitative method for determining RNA transcript levels in samples. Such methods include polymerase-based assays, such as RT-PCR, Taqman™, hybridization-based assays, for example using DNA microarrays or other solid support (e.g., Whole Genome DASL™ Assay, Illumina, Inc.), nucleic acid sequence based amplification (NASBA), flap endonuclease-based assays, as well as direct mRNA capture with branched DNA (QuantiGene™) or Hybrid Capture™ (Digene). The assay format, in addition to determining the gene expression profiles, will also allow for the control of, inter alia, intrinsic signal intensity variation between tests. Such controls may include, for example, controls for background signal intensity and/or sample processing, and/or other desirable controls for gene expression quantification across samples. For example, expression levels between samples may be controlled by testing for the expression level of one or more genes that are not associated with enriched pathways or differentially expressed between drug-sensitive and drug-resistant cells, or which are generally expressed at similar levels across the population. Such genes may include constitutively expressed genes, many of which are known in the art. Exemplary assay formats for determining gene expression levels, and thus for preparing gene expression profiles and drug-sensitive and drug-resistant signatures are described in this section.

In determining a tumor's gene expression profile, or in determining a drug-sensitive or drug-resistant profile in accordance with the invention, a hybridization-based assay may be employed. Nucleic acid hybridization involves contacting a probe and a target sample under conditions where the probe and its complementary target sequence (if present) in the sample can form stable hybrid duplexes through complementary base pairing. The nucleic acids that do not form hybrid duplexes may be washed away leaving the hybridized nucleic acids to be detected, typically through detection of an attached detectable label. It is generally recognized that nucleic acids may be denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In certain embodiments, hybridization is performed at low stringency, such as 6×SSPET at 37° C. (0.005% Triton X-100), to ensure hybridization, and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that may be present, as described below (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

The hybridized nucleic acids are typically detected by detecting one or more labels attached to the sample nucleic acids. The labels may be incorporated by any of a number of means well known to those of skill in the art. See WO 99/32660.

Numerous hybridization assay formats are known, and which may be used in accordance with the invention. Such hybridization-based formats include solution-based and solid support-based assay formats. Solid supports containing oligonucleotide probes designed to detect differentially expressed genes can be filters, polyvinyl chloride dishes, particles, beads, microparticles or silicon or glass based chips, etc. Any solid surface to which oligonucleotides can be bound, either directly or indirectly, either covalently or non-covalently, may be used. Bead-based assays are described, for example, in U.S. Pat. Nos. 6,355,431, 6,396,995, and 6,429,027, which are hereby incorporated by reference. Other chip-based assays are described in U.S. Pat. Nos. 6,673,579, 6,733,977, and 6,576,424, which are hereby incorporated by reference.

An exemplary solid support is a high density array or DNA chip, which may contain a particular oligonucleotide probes at predetermined locations on the array. Each predetermined location may contain more than one molecule of the probe, but each molecule within the predetermined location has an identical probe sequence. Such predetermined locations are termed features.

Oligonucleotide probe arrays for determining gene expression can be made and used according to any techniques known in the art (see for example, Lockhart et al (1996), Nat Biotechnol 14:1675-1680; McGall et al. (1996), Proc Nat Acad Sci USA 93:13555-13460). Such probe arrays may contain the oligonucleotide probes necessary for determining a tumor's gene expression profile, or for preparing drug-resistant and drug-sensitive signatures. Thus, such arrays may contain oligonucleotide designed to hybridize to at least 2, 3, 4, 5, 6, 7, 8, 9, 10, 20, 30, 50, 70, 100, 200, 300 or more of the genes described herein (e.g., as described in FIG. 3 or one of Tables 1-3). In some embodiments, the array contains probes designed to hybridize to all or nearly all of the genes listed in one or more of Tables 1-3. In still other embodiments, arrays are constructed that contain oligonucleotides designed to detect all or nearly all of the genes in Tables 1-3 on a single solid support substrate, such as a chip or a set of beads. The array, bead set, or probe set may contain, in some embodiments, no more than 3000 probes, no more than 2000 probes, no more than 1000 probes, or no more than 500 probes, so as to embody a custom probe set for determining gene expression signatures in accordance with the invention.

Probes based on the sequences of the genes described herein for preparing expression profiles may be prepared by any suitable method. Oligonucleotide probes, for hybridization-based assays, will be of sufficient length or composition (including nucleotide analogs) to specifically hybridize only to appropriate, complementary nucleic acids (e.g., exactly or substantially complementary RNA transcripts or cDNA). Typically the oligonucleotide probes will be at least about 10, 12, 14, 16, 18, 20 or 25 nucleotides in length. In some cases, longer probes of at least 30, 40, or 50 nucleotides may be desirable. In some embodiments, complementary hybridization between a probe nucleic acid and a target nucleic acid embraces minor mismatches (e.g., one, two, or three mismatches) that can be accommodated by reducing the stringency of the hybridization media to achieve the desired detection of the target polynucleotide sequence. Of course, the probes may be perfect matches with the intended target probe sequence, for example, the probes may each have a probe sequence that is perfectly complementary to a target sequence (e.g., a sequence of a gene listed in Tables 1-3).

A probe is a nucleic acid capable of binding to a target nucleic acid of complementary sequence through one or more types of chemical bonds, usually through complementary base pairing, usually through hydrogen bond formation. A probe may include natural (i.e., A, G, U, C, or T) or modified bases (7-deazaguanosine, inosine, etc.), or locked nucleic acid (LNA). In addition, the nucleotide bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages.

When using hybridization-based assays, in may be necessary to control for background signals. The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each location of the array. In an exemplary embodiment, background is calculated as the average hybridization signal intensity for the lowest 5% to 10% of the probes in the array. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all. Of course, one of skill in the art will appreciate that hybridization signals may be controlled for background using one or a combination of known approached, including one or a combination of approaches described in this paragraph.

The hybridization-based assay will be generally conducted under conditions in which the probe(s) will hybridize to their intended target subsequence, but with only insubstantial hybridization to other sequences or to other sequences, such that the difference may be identified. Such conditions are sometimes called “stringent conditions.” Stringent conditions are sequence-dependent and can vary under different circumstances. For example, longer probe sequences generally hybridize to perfectly complementary sequences (over less than fully complementary sequences) at higher temperatures. Generally, stringent conditions may be selected to be about 5° C. lower than the thermal melting point (Tm) for the specific sequence at a defined ionic strength and pH. Exemplary stringent conditions may include those in which the salt concentration is at least about 0.01 to 1.0 M Na+ ion concentration (or other salts) at pH 7.0 to 8.3 and the temperature is at least about 30° C. for short probes (e.g., 10 to 50 nucleotides). Desired hybridization conditions may also be achieved with the addition of agents such as formamide or tetramethyl ammonium chloride (TMAC).

When using an array, one of skill in the art will appreciate that an enormous number of array designs are suitable for the practice of this invention. The array will typically include a number of test probes that specifically hybridize to the sequences of interest. That is, the array will include probes designed to hybridize to any region of the genes listed in Tables 1-3. In instances where the gene reference in the Tables is an EST, probes may be designed from that sequence or from other regions of the corresponding full-length transcript that may be available in any of the public sequence databases, such as those herein described. See WO 99/32660 for methods of producing probes for a given gene or genes. In addition, software is commercially available for designing specific probe sequences. Typically, the array will also include one or more control probes, such as probes specific for a constitutively expressed gene, thereby allowing data from different hybridizations to be normalized or controlled.

The hybridization-based assays may include, in addition to “test probes” (e.g., that bind the target sequences of interest, which are listed in Tables 1-3), the assay may also test for hybridization to one or a combination of control probes. Exemplary control probes include: normalization controls, expression level controls, and mismatch controls. For example, when determining the levels of gene expression in patient or control samples, the expression values may be normalized to control between samples. That is, the levels of gene expression in each sample may be normalized by determining the level of expression of at least one constitutively expressed gene in each sample. In accordance with the invention, the constitutively expressed gene is generally not differentially expressed in drug-sensitive versus drug-resistant samples.

Other useful controls are normalization controls, for example, using probes designed to be complementary to a labeled reference oligonucleotide added to the nucleic acid sample to be assayed. The signals obtained from the normalization controls after hybridization provide a control for variations in hybridization conditions, label intensity, “reading” efficiency and other factors that may cause the signal of a perfect hybridization to vary between arrays. In one embodiment, signals (e.g., fluorescence intensity) read from all other probes in the array are divided by the signal (e.g., fluorescence intensity) from the control probes thereby normalizing the measurements. Exemplary normalization probes are selected to reflect the average length of the other probes (e.g., test probes) present in the array, however, they may be selected to cover a range of lengths. The normalization control(s) may also be selected to reflect the (average) base composition of the other probes in the array. In some embodiments, the assay employs one or a few normalization probes, and they are selected such that they hybridize well (i.e., no secondary structure) and do not hybridize to any potential targets.

The hybridization-based assay may employ expression level controls, for example, probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

The hybridization-based assay may also employ mismatch controls for the target sequences, and/or for expression level controls or for normalization controls. Mismatch controls are probes designed to be identical to their corresponding test or control probes, except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20-mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present, the perfect match probes should provide a more intense signal than the mismatch probes. The difference in intensity between the perfect match and the mismatch probe helps to provide a good measure of the concentration of the hybridized material.

Alternatively, the invention may employ reverse transcription polymerase chain reaction (RT-PCR), which is a sensitive method for the detection of mRNA, including low abundant mRNAs present in clinical samples. The application of fluorescence techniques to RT-PCR combined with suitable instrumentation has led to quantitative RT-PCR methods that combine amplification, detection and quantification in a closed system. Two commonly used quantitative RT-PCR techniques are the Taqman RT-PCR assay (ABI, Foster City, USA) and the Lightcycler assay (Roche, USA).

Thus, in one embodiment of the present invention, the preparation of patient gene expression profiles or the preparation of drug-sensitive and drug-resistant profiles comprises conducting real-time quantitative PCR (TaqMan) with sample-derived RNA and control RNA. Holland, et al., PNAS 88:7276-7280 (1991) describe an assay known as a Taqman assay. The 5′ to 3′ exonuclease activity of Taq polymerase is employed in a polymerase chain reaction product detection system to generate a specific detectable signal concomitantly with amplification. An oligonucleotide probe, non-extendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. A version of this assay is also described in Gelfand et al., in U.S. Pat. No. 5,210,015, which is hereby incorporated by reference.

Further, U.S. Pat. No. 5,491,063 to Fisher, et al., which is hereby incorporated by reference, provides a Taqman-type assay. The method of Fisher et al. provides a reaction that results in the cleavage of single-stranded oligonucleotide probes labeled with a light-emitting label wherein the reaction is carried out in the presence of a DNA binding compound that interacts with the label to modify the light emission of the label. The method of Fisher uses the change in light emission of the labeled probe that results from degradation of the probe.

The TaqMan detection assays offer certain advantages. First, the methodology makes possible the handling of large numbers of samples efficiently and without cross-contamination and is therefore adaptable for robotic sampling. As a result, large numbers of test samples can be processed in a very short period of time using the TaqMan assay. Another advantage of the TaqMan system is the potential for multiplexing. Since different fluorescent reporter dyes can be used to construct probes, the expression of several different genes associated with drug sensitivity or resistance may be assayed in the same PCR reaction, thereby reducing the labor costs that would be incurred if each of the tests were performed individually. Thus, the TaqMan assay format is preferred where the patient's gene expression profile, and the corresponding drug-sensitive and drug-resistance profiles comprise the expression levels of about 20 of fewer, or about 10 or fewer, or about 7 of fewer, or about 5 genes (e.g., genes listed in one or more of Tables 1-3.

### Diagnostic Kits and Probe Sets

The invention further provides a kit or probe array containing nucleic acid primers and/or probes for determining the level of expression in a patient tumor specimen or cell culture of a plurality of genes listed in Tables 1-3. The probe array may contain 3000 probes or less, 2000 probes or less, 1000 probes or less, 500 probes or less, so as to embody a custom set for preparing gene expression profiles described herein. In some embodiments, the kit may consist essentially of primers and/or probes related to evaluating drug-sensitivity/resistant in a sample, and primers and/or probes related to necessary or meaningful assay controls (such as expression level controls and normalization controls, as described herein under “Gene Expression Assay Formats”).

The kit for evaluating drug-sensitivity/resistance may comprise nucleic acid probes and/or primers designed to detect the expression level of ten or more genes associated with drug sensitivity/resistance, such as the genes listed in Tables 1-3. The kit may include a set of probes and/or primers designed to detect or quantify the expression levels of at least 5, 7, 10, or 20 genes listed in one of Tables 1-3. The primers and/or probes may be designed to detect gene expression levels in accordance with any assay format, including those described herein under the heading “Assay Format.” Exemplary assay formats include polymerase-based assays, such as RT-PCR, Taqman™, hybridization-based assays, for example using DNA microarrays or other solid support, nucleic acid sequence based amplification (NASBA), flap endonuclease-based assays. The kit need not employ a DNA microarray or other high density detection format.

In accordance with this aspect, the probes and primers may comprise antisense nucleic acids or oligonucleotides that are wholly or partially complementary to the diagnostic targets described herein (e.g., Tables 1-3). The probes and primers will be designed to detect the particular diagnostic target via an available nucleic acid detection assay format, which are well known in the art. The kits of the invention may comprise probes and/or primers designed to detect the diagnostic targets via detection methods that include amplification, endonuclease cleavage, and hybridization.

### EXAMPLES

### Example 1

### Methods

Forty-five breast cancer cells lines (Table 1) obtained from ATCC (Manassas, Va.) and DSMZ were maintained in culture in RPMI 1640 (Mediatech, Herndon, Va.) containing 10% FBS (HyClone, Logan, Utah) at 37° C. in 5% CO2.

An automated liquid handler (Dynamic Devices, Inc, Wilmington, Del.) was used to seed cells from each cell line into the wells of a 384-well microtiter plate. Cells were allowed to adhere to the plate and grown for 24 h at 37° C. in 5% CO₂. For each drug treatment, a liquid handler was used to prepare 10 serial dilutions in 10% RPMI 1640 in a 96-deep-well microtiter plate. The liquid handler was then used to add these 10 doses in triplicate to the adherent cell lines on 384-well plates. The drugs tested were common therapeutic agents, doxorubicin (A), paclitaxel (T), 5-fluorouracil (F), docetaxel (dT), epirubicin (E) (McKesson Specialty Care Solutions, La Vergne, Tenn.), and cyclophosphamide (C) (Niomech, Bielefeld, Germany) in the following drug combinations: (1) TFAC, (2) EC (3) FEC. In addition, 3 control wells for each drug combination contained cells with only medium. The cells were then incubated for 72 h (Gallion, et al., 2006; Kornblith, et al., 2004; Kornblith, et al., 2003; Ochs, et al., 2005). Medium and non-adherent (dead) cells were then removed from each well with the liquid handler. The remaining live cells were fixed in 95% ethanol and then stained with the DAPI (Molecular Probes, Eugene, Oreg.). A proprietary automated microscope (Precisions Therapeutics, Pittsburgh, Pa.) was used to capture UV images of the stained cells in each well, and the number of cells per well was counted for each well. For each dose of each drug combination, a survival fraction (SF) representing the ratio of cells that survived drug treatment was calculated based on the following formula: SF=Mean_{drug}/Mean_{control}, where Mean_{drug} is the average of the number of surviving cells of the three replicates at a dose, and Mean_{control} is the average number of cells remaining after 72 h incubation in the 3 control wells for each drug combination. The dose response cell survival curve was assumed to be monotonically decreasing; therefore the area under the curve (AUC) was used as an appropriate metric for the curve data reduction.

Gene expression data for each cell line were established by Hoeflich et al. 2009 by using Affymetrix HG-U133 Plus 2.0 chip and available at Gene Expression Omnibus (http://www.ncbi.nlm.nih.gov/geo/) with accession number GSE12777. The raw microarray data were processed by the software package RMA (Bolstad, et al., 2003; Irizarry, et al., 2003; Irizarry, et al., 2003) for the background adjustment and quantitative normalization. The processed data were log2-transformed. Non-specific gene filtering was performed to filter out probes whose interquartile range was less than median or had median expression values less than 100. Gene-wise and sample-wise normalization have been applied to filtered microarray datasets (Cheng, et al., 2009).

The C2 collection of MsigDB, a biology pathway database for cancer provided by Broad institute (Subramanian, et al., 2005) were used (v2.5 updated Apr. 7 2008).

The pathway analysis was similar to the method proposed by Adewale et al. (Adewale, et al., 2008). FIG. 1 shows a general diagram for pathway enrichment analysis in an individual microarray study.

1. Calculate t_{g}, the association score between gene g and yₛ, 1≦g≦G, where t_{g}=^{r}^{_{g}}/ₛ_{_{g}}.r_{g} is the linear regression coefficient between x_{gs and y}ₛ. 1≦s≦S. s_{g} is the standard error of r_{g.}

2. Compute vₚ the enrichment evidence score of pathway p, where:

$${V}_{p} = \frac{1}{G} {\sum }_{g = 1}^{G} {t}_{g} {z}_{gp}$$

3. Permute sample labels (AUC values) C times, and calculate the permuted statistics, Vₚ^{c}, 1≦c≦C.

4. Data standardization: suppose F₁, . . . , F_{G} are the empirical cumulative distribution functions of V_{g}, the data transformation function is:

φ_{g}(•)=Φ⁻¹{F_{g}(•)},g=1, . . . , G.

where Φ(•) is the cumulative distribution function for standard normal.

5. Estimate the p-value of pathway p as p(νₚ)=Σ_{c=1}^{C}Σ_{p′=1}^{P}I(V_{p′}^{c}≧Vₚ)/C•P and similarly calculate νₚ^{c}=Σ_{c′=1}^{C}Σ_{p′=1}^{P}I(V_{p′}^{c′}≧Pₚ^{c})/C•P Estimate π₀, the proportion of non-enriched pathways in the meta-analysis, as

$${\hat{π}}_{0} = \frac{{\sum }_{p = 1}^{P} I ( p ( {v}_{p} ) ∈ A )}{P \cdot l ( A )} .$$

Choose A=[0.5, 1] and thus l(A)=0.5. Estimate q-value of pathway p as:

q(νₚ)=\hat{π}₀Σ_{c=1}^{C}Σ_{p′=1}^{P}I(P_{p′}^{KS(c)}≦Pₚ^{KS})/C•Σ_{p′=1}^{P}I(P_{p′}^{KS}≦Pₚ^{KS}).

Pathways whose q-values are less than a pre-defined cutoff are considered as enriched pathways.

Suppose a data matrix {x_{gs}} (1≦g≦G, 1≦s≦S) represents the gene expression intensity of gene g and sample s. Let {yₛ} (1≦s≦S) represent the AUC for cell line s. A pathway database matrix {z_{gp}} (1≦g≦G, 1≦p≦P) represents the pathway information of P pathways, where z_{gp}=1 when gene g belongs to pathway p and z_{gp}=0 otherwise.

The pathway enrichment analysis has two main steps as follows.

Step I: The association scores with phenotype in each gene g are first calculated as t_{g}, where t_{g} is the correlation between gene expression values and AUC values.

Step II: The pathway enrichment evidence score νₚ is calculated for each pathway p. This is the key step in pathway enrichment analysis. The pathway enrichment evidence score is used to summarize the association scores of all genes in the pathway.

From the pathways determined to be related to drug response, TFAC, EC and FEC response, further genomic predictors were identified using supervised principal component regression. The identified TFAC genomic predictors were validated in predicting patients' pathologic complete response (pCR) for a public clinical trial with 133 breast cancer patients treated with TFAC. The identified EC genomic predictors were validated in predicting patients' pathologic complete response (pCR) for a public clinical trial with 37 breast cancer patients treated with EC. The identified FEC genomic predictors were validated in predicting patients' pathologic complete response (pCR) for a public clinical trial with 87 breast cancer patients treated with FAC.

Fold change values between sensitive and resistant cell lines were calculated by sorting the cell lines based on their AUC values. The top ⅓ of the cell lines are defined as sensitive and the bottom ⅓ of the cell lines are defined as resistant. For the fold change the calculation is done as follows for each gene: mean raw expression value for the drug sensitive group/mean raw expression value for the drug resistant group.

### Results

Enriched pathways are listed in FIG. 2. In FIG. 2, each row is the pre-defined pathway. Each column is the drug tested on breast cancer cell lines. “1” represents a pathway whose pvalue is less than 0.01, whereas 0 represents a pathway whose pvalue is larger than 0.01. 32 pathways were identified to be related to TFAC response, 32 pathways were identified to be related to EC response, 24 pathways were identified to be related to FAC response.

The pathway analyses are based on breast cancer cell lines instead of, for example, NCI60 cancer cell lines. NCI60 cancer cell lines are a mixture of breast, lung leukemia, colon, CNS, melanoma, ovarian, renal, and prostate cancer cell lines. The analyses on NCI60 cancer cell lines focus on common cancer pathways while the present analyses focuses on pathways particular for breast cancer.

These pathways are valuable for new biomarker identification, and new drug targeting, such as for discovering anthracycline drugs.

Genomic predictors (gene signatures) for sensitivity to TFAC, EC, and FEC were developed from the enriched pathways, and the top 50 genes associated with drug response are shown in Tables 1-3. Validation of these predictors produced the following results as illustrated in FIGS. 4-6.

FIG. 4 illustrates the accuracy of a 50-gene signature from Table for predicting pCR in an independent patient population (133 neoadjuvant breast cancer patients treated with TFAC). Outcome is pathological complete response (pCR). The results are shown as a receiver operator curve (ROC). When using one third of the prediction scores as cutoff, the accuracy is 0.73, sensitivity is 0.62 and specificity is 0.78. The right panel shows that the gene expression signature of Table 1 is stable over a large range of increasing gene number, from less than about 10 to over 350 genes (Table 1 lists the top 50 genes).

FIG. 5 illustrates the accuracy of a 50-gene signature from Table 2 for predicting pCR in an independent patient population (37 neoadjuvant breast cancer patients treated with EC). Outcome is pathological complete response (pCR). The results are shown as a receiver operator curve (ROC). When using one third of the prediction scores as cutoff, the accuracy is 0.71, sensitivity is 0.56 and specificity is 0.77.The right panel shows that the gene expression signature of Table 3 is stable over a large range of increasing gene number, from less than about 10 to over 250 genes (Table 2 lists the top 50 genes).

FIG. 6 illustrates the accuracy of a 50-gene signature from Table 3 for predicting pCR in an independent patient population (87 neoadjuvant breast cancer patients treated with FAC). Outcome is pathological complete response (pCR). The results are shown as a receiver operator curve (ROC). When using one third of the prediction scores as cutoff, the accuracy is 0.71, sensitivity is 0.71 and specificity is 0.71. The right panel shows that the gene expression signature of Table 3 is stable over a large range of increasing gene number, from less than about 10 to over 350 genes (Table 3 lists the top 50 genes).

TABLE 1
TFAC

| | | | | fold |
|---|---|---|---|---|
| probeID | Gene. symbol | mean_sens | mean_resis | change |
| 208636_at | ACTN1 | 9503.45 | 5275.96 | 1.80 |
| 205260_s_at | ACYP1 | 1281.13 | 650.92 | 1.97 |
| 202381_at | ADAM9 | 5335.71 | 3215.14 | 1.66 |
| 213702_x_at | ASAH1 | 5070.38 | 8020.90 | 0.63 |
| 203968_s_at | CDC6 | 3602.75 | 1323.52 | 2.72 |
| 203492_x_at | CEP57 | 1278.45 | 806.81 | 1.58 |
| 221223_x_at | CISH | 856.38 | 1640.33 | 0.52 |
| 228496_s_at | CRIM1 | 2346.20 | 723.25 | 3.24 |
| 201533_at | CTNNB1 | 4000.32 | 2347.66 | 1.70 |
| 202613_at | CTPS | 2578.65 | 1576.45 | 1.64 |
| 223421_at | CYHR1 | 785.52 | 1497.58 | 0.52 |
| 225078_at | EMP2 | 3835.58 | 7348.03 | 0.52 |
| 227017_at | ERICH1 | 637.58 | 458.22 | 1.39 |
| 203282_at | GBE1 | 3538.89 | 1289.35 | 2.74 |
| 212335_at | GNS | 2956.75 | 3764.39 | 0.79 |
| 225988_at | HERC4 | 1733.63 | 1117.44 | 1.55 |
| 215071_s_at | HIST1H2AC | 1668.20 | 3410.31 | 0.49 |
| 209911_x_at | HIST1H2BD | 2969.48 | 4450.72 | 0.67 |
| 209806_at | HIST1H2BK | 8187.66 | 11562.07 | 0.71 |
| 210189_at | HSPA1L | 85.83 | 154.35 | 0.56 |
| 201631_s_at | IER3 | 11146.28 | 5666.34 | 1.97 |
| 212473_s_at | MICAL2 | 2516.31 | 639.26 | 3.94 |
| 202431_s_at | MYC | 4130.85 | 2108.33 | 1.96 |
| 214440_at | NAT1 | 923.22 | 3415.33 | 0.27 |
| 203045_at | NINJ1 | 1384.50 | 2192.21 | 0.63 |
| 204088_at | P2RX4 | 692.84 | 1469.26 | 0.47 |
| 209494_s_at | PATZ1 | 839.60 | 2062.89 | 0.41 |
| 212593_s_at | PDCD4 | 3277.77 | 8409.78 | 0.39 |
| 204613_at | PLCG2 | 318.58 | 178.98 | 1.78 |
| 209633_at | PPP2R3A | 1216.06 | 706.43 | 1.72 |
| 202187_s_at | PPP2R5A | 1461.19 | 2245.91 | 0.65 |
| 213093_at | PRKCA | 1084.31 | 286.50 | 3.78 |
| 218379_at | RBM7 | 1979.96 | 1190.61 | 1.66 |
| 212099_at | RHOB | 5264.00 | 11427.60 | 0.46 |
| 212724_at | RND3 | 4737.53 | 1814.69 | 2.61 |
| 202636_at | RNF103 | 2353.81 | 4327.87 | 0.54 |
| 209339_at | SIAH2 | 1749.38 | 3882.41 | 0.45 |
| 205074_at | SLC22A5 | 1059.54 | 2090.21 | 0.51 |
| 222529_at | SLC25A37 | 823.71 | 301.42 | 2.73 |
| 201349_at | SLC9A3R1 | 5412.87 | 11932.24 | 0.45 |
| 235020_at | TAF4B | 522.44 | 176.54 | 2.96 |
| 212956_at | TBC1D9 | 2028.14 | 4725.61 | 0.43 |
| 201764_at | TMEM106C | 3642.59 | 5476.92 | 0.67 |
| 208296_x_at | TNFAIP8 | 917.61 | 654.27 | 1.40 |
| 228834_at | TOB1 | 5785.91 | 11778.08 | 0.49 |
| 204485_s_at | TOM1L1 | 1726.56 | 3784.39 | 0.46 |
| 208763_s_at | TSC22D3 | 2713.04 | 5468.80 | 0.50 |
| 200931_s_at | VCL | 6847.25 | 3793.12 | 1.81 |
| 202908_at | WFS1 | 1040.38 | 2069.40 | 0.50 |
| 200670_at | XBP1 | 8775.99 | 16074.90 | 0.55 |

TABLE 2
EC

| | | | | fold |
|---|---|---|---|---|
| probeID | Gene. symbol | mean_sens | mean_resis | change |
| 205260_s_at | ACYP1 | 1323.32 | 610.87 | 2.17 |
| 202381_at | ADAM9 | 5866.12 | 3089.33 | 1.90 |
| 205891_at | ADORA2B | 2090.99 | 575.67 | 3.63 |
| 205047_s_at | ASNS | 4700.07 | 2073.97 | 2.27 |
| 209464_at | AURKB | 2144.91 | 1046.97 | 2.05 |
| 202870_s_at | CDC20 | 5556.95 | 2968.47 | 1.87 |
| 201853_s_at | CDC25B | 4543.44 | 3130.06 | 1.45 |
| 203968_s_at | CDC6 | 2906.15 | 1239.65 | 2.34 |
| 203492_x_at | CEP57 | 1402.35 | 844.27 | 1.66 |
| 228496_s_at | CRIM1 | 2737.66 | 765.36 | 3.58 |
| 221139_s_at | CSAD | 406.77 | 835.69 | 0.49 |
| 202613_at | CTPS | 2749.79 | 1546.23 | 1.78 |
| 223421_at | CYHR1 | 655.85 | 1484.84 | 0.44 |
| 225078_at | EMP2 | 3306.47 | 7221.25 | 0.46 |
| 203499_at | EPHA2 | 1503.07 | 335.91 | 4.47 |
| 1438_at | EPHB3 | 450.68 | 1038.09 | 0.43 |
| 227017_at | ERICH1 | 681.42 | 460.57 | 1.48 |
| 203282_at | GBE1 | 3219.53 | 1203.89 | 2.67 |
| 221510_s_at | GLS | 2261.72 | 1446.89 | 1.56 |
| 212335_at | GNS | 3000.46 | 3785.92 | 0.79 |
| 225988_at | HERC4 | 2123.97 | 1143.30 | 1.86 |
| 215071_s_at | HIST1H2AC | 1712.22 | 3369.28 | 0.51 |
| 209911_x_at | HIST1H2BD | 2944.21 | 4464.40 | 0.66 |
| 210189_at | HSPA1L | 72.85 | 159.79 | 0.46 |
| 201631_s_at | IER3 | 10520.90 | 5496.12 | 1.91 |
| 212473_s_at | MICAL2 | 2658.14 | 676.75 | 3.93 |
| 202431_s_at | MYC | 4982.95 | 1939.31 | 2.57 |
| 201976_s_at | MYO10 | 2730.90 | 1091.11 | 2.50 |
| 214440_at | NAT1 | 952.57 | 3487.98 | 0.27 |
| 218086_at | NPDC1 | 1446.00 | 3330.89 | 0.43 |
| 200790_at | ODC1 | 6653.43 | 2375.73 | 2.80 |
| 204088_at | P2RX4 | 651.16 | 1510.25 | 0.43 |
| 210448_s_at | P2RX5 | 720.63 | 161.36 | 4.47 |
| 209494_s_at | PATZ1 | 803.86 | 2242.86 | 0.36 |
| 212593_s_at | PDCD4 | 3151.73 | 8722.83 | 0.36 |
| 203554_x_at | PTTG1 | 9631.34 | 6301.51 | 1.53 |
| 218379_at | RBM7 | 2123.97 | 1201.65 | 1.77 |
| 212099_at | RHOB | 4516.38 | 11793.16 | 0.38 |
| 212724_at | RND3 | 5203.60 | 1844.69 | 2.82 |
| 202636_at | RNF103 | 2133.44 | 4237.30 | 0.50 |
| 212590_at | RRAS2 | 3375.01 | 1046.82 | 3.22 |
| 204502_at | SAMHD1 | 363.27 | 220.50 | 1.65 |
| 209339_at | SIAH2 | 1776.11 | 4351.41 | 0.41 |
| 222529_at | SLC25A37 | 647.74 | 311.97 | 2.08 |
| 201349_at | SLC9A3R1 | 5226.02 | 12356.77 | 0.42 |
| 235020_at | TAF4B | 498.15 | 184.71 | 2.70 |
| 201764_at | TMEM106C | 3499.52 | 5316.15 | 0.66 |
| 228834_at | TOB1 | 5403.69 | 11177.02 | 0.48 |
| 202908_at | WFS1 | 1159.85 | 1907.34 | 0.61 |
| 200670_at | XBP1 | 7838.42 | 16253.14 | 0.48 |

TABLE 3
FEC

| | | | | fold |
|---|---|---|---|---|
| probeID | Gene. symbol | mean_sens | mean_resis | change |
| 205260_s_at | ACYP1 | 1376.54 | 657.31 | 2.09 |
| 202381_at | ADAM9 | 6281.84 | 2993.47 | 2.10 |
| 205891_at | ADORA2B | 2333.75 | 438.73 | 5.32 |
| 219806_s_at | C11ORF75 | 923.27 | 737.67 | 1.25 |
| 34726_at | CACNB3 | 570.77 | 1172.46 | 0.49 |
| 202870_s_at | CDC20 | 5503.72 | 2957.35 | 1.86 |
| 201853_s_at | CDC25B | 5380.42 | 3098.56 | 1.74 |
| 203968_s_at | CDC6 | 3583.54 | 1413.45 | 2.54 |
| 203492_x_at | CEP57 | 1411.68 | 854.75 | 1.65 |
| 201533_at | CTNNB1 | 3623.35 | 2404.15 | 1.51 |
| 202613_at | CTPS | 3097.18 | 1619.78 | 1.91 |
| 225078_at | EMP2 | 3194.45 | 7222.13 | 0.44 |
| 203499_at | EPHA2 | 1769.91 | 334.49 | 5.29 |
| 1438_at | EPHB3 | 532.04 | 1034.20 | 0.51 |
| 227017_at | ERICH1 | 710.74 | 464.84 | 1.53 |
| 203725_at | GADD45A | 3276.57 | 817.76 | 4.01 |
| 203282_at | GBE1 | 3487.51 | 1162.12 | 3.00 |
| 225988_at | HERC4 | 2358.75 | 1164.17 | 2.03 |
| 210189_at | HSPA1L | 79.34 | 160.76 | 0.49 |
| 201631_s_at | IER3 | 10331.43 | 5418.61 | 1.91 |
| 204626_s_at | ITGB3 | 147.15 | 95.38 | 1.54 |
| 213358_at | KIAA0802 | 1098.80 | 349.23 | 3.15 |
| 225611_at | MAST4 | 403.20 | 1044.64 | 0.39 |
| 212473_s_at | MICAL2 | 2749.17 | 684.00 | 4.02 |
| 202431_s_at | MYC | 4835.18 | 2341.63 | 2.06 |
| 201976_s_at | MYO10 | 2899.00 | 1084.59 | 2.67 |
| 203045_at | NINJ1 | 1151.48 | 2113.26 | 0.54 |
| 205005_s_at | NMT2 | 720.23 | 308.98 | 2.33 |
| 204088_at | P2RX4 | 642.48 | 1458.64 | 0.44 |
| 209494_s_at | PATZ1 | 794.11 | 2193.46 | 0.36 |
| 212593_s_at | PDCD4 | 2979.21 | 8389.55 | 0.36 |
| 202738_s_at | PHKB | 1501.16 | 2647.97 | 0.57 |
| 204613_at | PLCG2 | 284.06 | 184.01 | 1.54 |
| 207000_s_at | PPP3CC | 293.30 | 118.33 | 2.48 |
| 213093_at | PRKCA | 1309.20 | 299.35 | 4.37 |
| 218379_at | RBM7 | 2282.65 | 1217.14 | 1.88 |
| 212120_at | RHOQ | 2463.82 | 1377.37 | 1.79 |
| 212724_at | RND3 | 5350.64 | 1856.36 | 2.88 |
| 202636_at | RNF103 | 2254.86 | 3984.62 | 0.57 |
| 212590_at | RRAS2 | 2952.87 | 968.49 | 3.05 |
| 209339_at | SIAH2 | 1559.23 | 4253.10 | 0.37 |
| 205074_at | SLC22A5 | 1079.75 | 2072.45 | 0.52 |
| 222529_at | SLC25A37 | 873.01 | 311.03 | 2.81 |
| 209884_s_at | SLC4A7 | 1256.12 | 628.73 | 2.00 |
| 235020_at | TAF4B | 451.72 | 183.08 | 2.47 |
| 201764_at | TMEM106C | 3342.02 | 5387.71 | 0.62 |
| 208296_x_at | TNFAIP8 | 1150.41 | 655.26 | 1.76 |
| 228834_at | TOB1 | 4937.37 | 12047.00 | 0.41 |
| 200670_at | XBP1 | 8132.33 | 15802.44 | 0.51 |
| 202932_at | YES1 | 4345.60 | 2541.55 | 1.71 |

### REFERENCES

All references and databases cited herein, including those identified below, are hereby incorporated by reference.

Adewale, A. J., et al. (2008) Pathway analysis of microarray data via regression, J Comput Biol, 15, 269-277.

Bolstad, B. M., et al. (2003) A comparison of normalization methods for high density oligonucleotide array data based on variance and bias, Bioinformatics, 19, 185-193.

Cheng, C., et al. (2009) Ratio adjustment and calibration scheme for gene-wise normalization to enhance microarray inter-study prediction, Bioinformatics, 25, 1655-1661.

Gallion, H., et al. (2006) Progression-free interval in ovarian cancer and predictive value of an ex vivo chemoresponse assay, Int J Gynecol Cancer, 16, 194-201.

Hoeflich K P, et al (2009) In vivo Antitumor Activity of MEK and Phosphatidylinositol 3-Kinase Inhibitors in Basal-Like Breast Cancer Models. Clinical Cancer Research, 15(14):4649-4664.

Irizarry, R. A., et al. (2003) Summaries of Affymetrix GeneChip probe level data, Nucleic Acids Res, 31, el5.

Irizarry, R. A., et al. (2003) Exploration, normalization, and summaries of high density oligonucleotide array probe level data, Biostatistics, 4, 249-264.

Kornblith, P., et al. (2004) Differential in vitro effects of chemotherapeutic agents on primary cultures of human ovarian carcinoma, Int J Gynecol Cancer, 14, 607-615.

Kornblith, P., et al. (2003) In vitro responses of ovarian cancers to platinums and taxanes, Anticancer Res, 23, 543-548.

Ochs, R. L., Burholt, D. and Kornblith, P. (2005) The ChemoFx assay: an ex vivo cell culture assay for predicting anticancer drug responses, Methods Mol Med, 110, 155-172.

Subramanian, A., et al. (2005) Gene set enrichment analysis: A knowledge-based approach for interpreting genome-wide expression profiles, Proceedings of the National Academy of Sciences of the United States of America, 102, 15545-15550.

1. A method for determining a drug response profile for a breast tumor specimen or a cell culture derived therefrom, comprising:
extracting or isolating RNA for the breast tumor specimen or culture derived therefrom, and
and determining the expression level of genes associated with enriched pathways in drug sensitive or drug resistant cells, to thereby prepare a drug response profile.

2. The method of claim 1, wherein the tumor specimen is a surgical specimen.

3. (canceled)

4. The method of claim 1, wherein the patient is a candidate for treatment with one or more of paclitaxel, 5-fluorouracil, doxorubicin, cyclophosphamide, epirubicin or combinations thereof.

5. (canceled)

6. The method of claim 1, wherein the RNA is extracted or isolated from the tumor specimen.

7. The method of claim 1, wherein RNA is extracted or isolated from a culture derived from the tumor specimen, the culture being enriched for malignant cells versus stromal cells.

8. The method of claim 7, wherein the culture is a monolayer grown from tumor explants prepared substantially by mechanical fragmentation.

9. The method of claim 1, wherein the gene expression profile is generated using a polynucleotide hybridization assay.

10. The method of claim 9, wherein the hybridization assay is microarray analysis.

11. The method of claim 10, wherein the microarray comprises corresponding probes from the HG-U133 chip.

12. The method of claim 9, wherein the profile is generated using a polymerase-based assay.

13. The method of claim 12, wherein the assay is Real Time PCR.

14. The method of claim 1, wherein the profile comprises gene expression levels for the genes associated with the enriched pathways listed in FIG. 3.

15. The method of claim 14, wherein the profile comprises gene expression levels for genes associated with pathways enriched in TFAC sensitive-cells, the TFAC enriched pathways being listed in FIG. 3.

16. The method of claim 14, wherein the profile comprises gene expression levels for genes associated with pathways enriched in EC sensitive-cells, the EC enriched pathways being listed in FIG. 3.

17. The method of claim 14, wherein the profile comprises gene expression levels for genes associated with pathways enriched in FEC sensitive-cells, the FEC enriched pathways being listed in FIG. 3.

18. The method of claim 14, wherein the profile comprises at least 10 genes listed in one of Tables 1-3.

19. The method of claim 14, wherein the gene expression profile comprises the expression levels for at least about 100 genes, these genes being associated with the enriched pathways.

20. (canceled)

21. (canceled)

22. The method of claim 18, wherein the profile comprises the expression level of at least 20 genes listed in one of Tables 1-3.

23. The method of claim 22, wherein the profile comprises the expression level of at least 40 genes listed in one of Tables 1-3.

24. The method of claim 22, wherein the profile comprises the expression level for the genes listed in one or more of Tables 1-3.

25-28. (canceled)

29. The method of claim 1, wherein the gene expression profile is evaluated for the presence or absence of a pathway signature in FIG. 3.

30. The method of claim 29, wherein the pathway signature is indicative of sensitivity to one or more of the chemotherapeutics selected from the group consisting of TFAC, EC, and FEC.

31. (canceled)

32. (canceled)

33. The method of claim 1, wherein the gene expression profile is evaluated for the presence or absence of a gene expression signature of at least one of Tables 1-3.

34. The method of claim 33, wherein the gene expression signature is indicative of sensitivity to one or more of the chemotherapeutics selected from the group consisting of TFAC, EC and FEC.

35. (canceled)

36. (canceled)

37. The method of claim 33, wherein the signature comprises mean or median expression levels for drug sensitive and/or drug resistant cells.

38. The method of claim 29, further comprising, classifying the profile as sensitive or resistant to a drug or combination of drugs.

39. The method of claim 1, further comprises, conducting in vitro chemoresponse testing for the tumor specimen.

40. The method of claim 1, wherein the method is predictive of pathological complete response.

42. A method for identifying a pathway signature indicative of a breast cancer cell or cell line's sensitivity or resistance against a chemotherapeutic agent, comprising:
determining the level of sensitivity of a panel of breast cancer cell lines for the chemotherapeutic agent in vitro, and evaluating the gene expression levels of the breast cancer cell lines to identify biochemical pathways associated with the level of sensitivity.

43. The method of claim 42, wherein the panel of breast cancer cell lines are immortalized cell lines.

44. The method of claim 42, wherein the panel of breast cancer cell lines are derived from explants of patient tumor specimens.

45. A diagnostic kit or probe array comprising nucleic acid primers and/or probes for determining the level of expression in a patient tumor specimen or cell culture of a plurality of genes listed in one of Tables 1-3.

46. (canceled)
